(19) United States
(12) Patent Application Publication
(10) Pub. No.: US 2019/0390259 A1
(43) Pub. Date: Dec. 26, 2019

(54) COMPOSITIONS FOR AND METHODS OF ENRICHING GENETIC MUTANTS FOR DETECTING, DIAGNOSING, AND PROGNOSING CANCER

(71) Applicant: Lariat Biosciences, Inc., Beverly, MA (US)

(72) Inventor: Jonathan W. Larson, Chelsea, MA (US)

(21) Appl. No.: 16/091,858

(22) PCT Filed: Apr. 5, 2017
(86) PCT No.: PCT/US2017/026110

§ 371 Date: Oct. 5, 2018

Related U.S. Application Data
(60) Provisional application No. 62/318,532, filed on Apr. 5, 2016.

(51) Int. Cl. C12Q 1/6827; C12Q 1/6886

(52) U.S. Cl. CPC C12Q 1/6827; C12Q 1/6886; C12Q 2600/112; C12Q 2600/118; C12Q 2600/156

(57) ABSTRACT

Compositions for detecting, diagnosing and prognosing cancer in individuals having or suspected of having cancer are provided. Said compositions are used to enrich a nucleic acid target comprising a locus of genetic variation, e.g., single nucleotide polymorphism (SNPs) and variable mutations, such as small insertions, deletions, and replacements (“indels”) within a sample for ease and improved detection. In addition, kits are provided for measuring levels or the presence of SNPs and indels associated with cancer for detecting, diagnosing and prognosing cancer. Furthermore, methods are provided for detecting, diagnosing and prognosing cancer in individuals having or suspected of having cancer comprising determining the enrichment levels and/or presence or absence of the SNPs and indels in a subject.

### CROSS-REFERENCE

This application is a national stage filing under 35 U.S.C. § 371 of PCT/US17/26110, filed on Apr. 5, 2017, which claims the benefit of U.S. Provisional Application No. 62/318,532, filed Apr. 5, 2016. The entirety of these applications are hereby incorporated by reference in their entirety.

### FIELD OF THE INVENTION

The invention generally relates to medical diagnostics and in particular relates to compositions for and methods of detecting, diagnosing, or prognosing an individual having or suspected of having a cancer.

### BACKGROUND

Diagnosis of cancer from genetic biomarkers in the bloodstream—the liquid biopsy—has emerged as a powerful surrogate or replacement technique for invasive needle biopsies and tumor resections. Liquid biopsies mine genetic information from a variety of different sources including circulating free DNA, exosomes, and circulating tumor cells. However, in general the underlying biomarker is a mutation in genomic DNA, and the mutant molecules are strongly outnumbered by wild-type molecules by the thousands or by the millions. Accordingly, there is a great need in the art to enrich mutant molecules while diminishing the wild-type background for detecting, diagnosing, or prognosing cancer.

### BRIEF SUMMARY

The invention described here is both a method for genotyping mutant DNA amidst a vast abundance of wild-type DNA, regardless of the source, as well as a method for enriching mutant DNA or a nucleic acid target comprising a locus of genetic variation, e.g., known single nucleotide polymorphism (SNPs) and variable mutations, such as small insertions, deletions, and replacements (“indels”). Benefits of the invention will include the detection of cancer at earlier stages when patient prognosis is much more favorable.

Compositions are provided for detecting, diagnosing and prognosing cancer in an individual having or suspected of having a cancer. Said compositions are used to enrich SNPs and indels significantly and abundantly over wild-type levels for ease and improved detection. In one aspect of the invention, the composition can be a kit for detecting, diagnosing and/or prognosing cancer, the kit having a plurality of probes and/or nucleotide primer pairs, where each of the probes or nucleotide primer pairs specifically binds to at least one distinct or plurality of SNP or indel.

Methods are also provided for detecting, diagnosing and prognosing cancer and predicting the likelihood of metastasis in an individual having or suspected of having a cancer. In one aspect, the method can include measuring the levels of at least one, or a plurality, of SNPs and indels in a sample from an individual having or suspected of having a cancer where the SNPs and indels are enriched when compared to a control/reference or wild-type levels.

The compositions and methods therefore find use in detecting, predicting and diagnosing an early stage cancer, as well as find use in prognosing an individual having cancer, which can be used to determine an appropriate treatment regimen.

These and other features, objects and advantages of the present invention will become better understood from the description that follows. In the description, reference is made to the accompanying drawings, which form a part hereof and in which there is shown by way of illustration, not limitation, embodiments of the invention.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1 shows a diagram of genotype enrichment where a primer extension assay incorporates a chain-terminating and base-pair specific moiety, preferably a dideoxynucleotide, at the site of wild-type bases within the locus of a targeted mutation.

FIG. 2 shows single nucleotide polymorphism (SNP) genotyping of DNA within hydrogel microparticles detected by epifluorescence microscopy using double stranded DNA intercalator YoYo-1. FIGS. 2A and 2B show the original DNA-impregnated particles, with bright fluorescence for both the wild-type and mutant. To demonstrate both (1) that the single-stranded DNA that melted off of the particles is mobile and can diffuse from within the particles, and (2) that the remaining bound DNA is enzyme-accessible throughout the particles, the DNA was digested by exonuclease immediately after the first melting step in the genotyping procedure. The fluorescent signal almost completely disappeared, confirming biochemical activity of the bound DNA (FIGS. 2 C and 2D). FIGS. 2E and 2F show the recovery of fluorescence after the full genotyping assay in the mutant-type particles, but not in the wild-type particles that remained at background values.

FIG. 3 shows bridge-mode droplet generation (as described in U.S. patent application Ser. No. 14/777,203.

FIG. 4 shows indel enrichment.

FIG. 5 shows indel genotyping.

FIG. 6 shows histograms of the log of the green fluorescence intensities of particles with wild-type and mutant DNA. Each plot describes particles in one of four different states of the assay. The “original” particles were quantified for fluorescence before the first cleaning step, a simple positive control indicating the detectable presence of DNA. “Melt, digest” particles were cleaned particles with the secondary strand melted away (a.k.a. ssDNA particles, above) that were then treated with exonuclease digestion. This negative control sample represents the minimum fluorescence because the exonuclease digests all of the ssDNA remaining after the melt. “Melt, extend, digest” particles are the second positive control revealing the maximum signal that can be regained through extension after the melt. Lastly the “full assay” particles are those described in the full protocol for this example, above.

While the invention is susceptible to various modifications and alternative forms, exemplary embodiments thereof are shown by way of example in the drawings and are described in detail below. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments herein and appended claims. Reference therefore should be made to the embodiments herein and appended claims for interpreting the scope of the invention.

### DETAILED DESCRIPTION OF PREFERRED EMBODIMENTS

The compositions and methods now will be described more fully hereinafter with reference to the accompanying drawings, in which some, but not all embodiments are shown.

Although specific terms are employed herein, they are used in a generic and descriptive sense only and not for purposes of limitation. As such, unless defined otherwise, all technical and scientific terms used herein have the same meaning as commonly understood by one of skill in the art to which the invention pertains. Although any materials and methods similar to or equivalent to those described herein can be used in the practice or testing of the invention, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “altered amount” of a marker or “altered level” of a marker refers to increased or decreased copy number of the SNPs or indels and/or increased or decreased nucleic acid level of a particular SNP or indel in a cancer sample, as compared to the level or copy number of the SNP or indel in a control sample or as compared to wild-type levels within the sample.

The term “altered level of expression” of a SNP or indel refers to an expression level or copy number of a SNP or indel in a test sample e.g., a sample derived from a subject suffering from cancer, that is greater or less than the control of the assay employed to assess expression or copy number, and may be at least 1.1, and in some embodiments may be 1.2, 1.3, 1.4, 1.5, 1.6, 1.7, 1.8, 1.9, two, three, four, five or ten or more times the expression level or copy number of the SNP or indel in a control sample (e.g., sample from a healthy subject not having the associated disease) and, in some embodiments, the average expression level or copy number of the SNP or indel in several control samples. The altered level of expression is greater or less than the standard error of the assay employed to assess expression or copy number, and is, in some embodiments, at least 1.1, and, in some embodiments may be 1.2, 1.3, 1.4, 1.5, 1.6, 1.7, 1.8, 1.9, two, three, four, five or ten or more times the expression level or copy number of the SNP or indel in a control sample (e.g., sample from a healthy subject not having the associated disease) and, in some embodiments may be, the average expression level or copy number of the SNP or indel in several control samples.

SNP or indel refers to the presence of mutations or allelic variants within a gene, e.g., mutations which affect expression or activity of the gene, as compared to the normal or wild-type gene. For example, indel mutations or variable mutations include, but are not limited to substitutions, deletions, replacement, insertions, or addition mutations. Such mutations may be present in the coding or non-coding region of the gene.

The “amount” of a SNP or indel, e.g., expression or copy number of a gene in a subject is “significantly” higher or lower than the normal amount of a wild-type gene, if the amount of the SNP or indel is greater or less, respectively, than the normal level by an amount greater than the control of the assay employed to assess amount, and, in some embodiments may be, at least 1.1, and, in some embodiments may be, 1.2, 1.3, 1.4, 1.5, 1.6, 1.7, 1.8, 1.9, two, three, four, five, ten or more times that amount. Alternately, the amount of the SNP or indel in the subject can be considered “significantly” higher or lower than the normal amount if the amount is at least about two, and in some embodiments may be at least about three, four, or five times, higher or lower, respectively, than the normal amount of the wild-type gene.

The terms “amplification” or “amplify” include the reactions necessary to increase the number of copies of a nucleic acid sequence (e.g., a DNA sequence). For the purposes of this invention, amplification refers to the in vitro exponential increase in copy number of a target nucleic acid sequence, such as that mediated by a polymerase amplification reaction such as, e.g., PCR, however, other amplification reactions encompassed by the invention include, e.g., RT-PCR (see, e.g., U.S. Pat. No. 4,683,202; Mullis et al.), and the ligase chain reaction (Barany, Proc. Natl. Acad. Sci. USA 88:189-193 (1991)).

The amplicons may be entrapped in hydrogel microparticles, microspheres, microbeads, or nanoparticles or affixed to a solid substrate for ease of optical detection or any known methods of detection set forth infra. In some embodiments, the amplicons or nucleic acid targets are attached to streptavidin-coaded microparticles. These may include DynaBead described at: https://www.thermofisher.com/us/en/home/brands/product-brand/dynal/streptavidin-coupled-dynabeads.html. In some embodiments, the solid substrate may be a material that may be modified to contain discrete individual sites appropriate for the attachment or association of the amplicons and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing. The substrate may be planar, although other configurations of substrates may be used as well. For example, amplicons may be placed on the inside surface of a tube, for flow-through sample analysis to minimize sample volume. Similarly, the substrate may be flexible, such as a flexible foam, including closed cell foams made of particular plastics. The support may be derivatized with chemical functional groups for subsequent attachment of the two. For example, the support may be derivatized with a chemical functional group including, but not limited to, amino groups, carboxyl groups, oxo groups or thiol groups. Using these functional groups, the amplicons may be attached using functional groups on the probes used to amplify either directly or indirectly using a linker. The amplicons may be attached to the solid support by either the 5′ terminus, 3′ terminus, or via an internal nucleotide. The amplicon may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit).

The term “cancer” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

Additional non-limiting examples of cancers include, hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, Wilms tumor.

The term “control” refers to any reference standard suitable to provide a comparison to the SNP or indel products in the test sample. In one embodiment, the control comprises obtaining a “control sample” from which wild-type product levels are detected and compared to the wild-type product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment. It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, for example, standard of care therapy for cancer, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), patients undergoing cancer therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The term “diagnosing cancer” includes the use of the methods, systems, and code of the present invention to determine the presence or absence of a cancer or subtype thereof in an individual. The term also includes methods, systems, and code for assessing the level of disease activity in an individual.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

The term “gene expression data” or “gene expression level” as used herein refers to information regarding the relative or absolute level of expression of a gene or set of genes in a cell or group of cells. The level of expression of a gene may be determined based on the level of genomic DNA, chromosomal DNA, RNA, such as mRNA, encoded by the gene. Gene expression data may be acquired for an individual cell, or for a group of cells such as a tumor or biopsy sample. Gene expression data and gene expression levels can be stored on computer readable media, e.g., the computer readable medium used in conjunction with a microarray or chip reading device. Such gene expression data can be manipulated to generate gene expression signatures.

The term “gene expression signature” or “signature” as used herein refers to a group of coordinately expressed genes. The genes making up this signature may be expressed in a specific cell lineage, stage of differentiation, or during a particular biological response. The genes can reflect biological aspects of the tumors in which they are expressed, such as the cell of origin of the cancer, the nature of the non-malignant cells in the biopsy, and the oncogenic mechanisms responsible for the cancer or pathology thereof (Shaffer et al., Immunity, 15: 375-385 (2001)).

As used herein, the term “inhibit” includes the decrease, limitation, or blockage, of, for example a particular action, function, or interaction. For example, cancer is “inhibited” if at least one symptom of the cancer, such as hyperproliferative growth, is alleviated, terminated, slowed, or prevented. As used herein, cancer is also “inhibited” if recurrence or metastasis of the cancer is reduced, slowed, delayed, or prevented.

The term “label” refers to a molecular moiety capable of detection including, by way of example, without limitation, radioactive labels which can be incorporated by known methods (e.g., nick translation or kinasing), radioactive isotopes, biotin, fluorescent groups, chemiluminescent groups (e.g., dioxetanes, particularly triggered dioxetanes), digoxigenin, enzymes, antibodies, luminescent agents, precipitating agents, dyes, and the like.

The “normal” level of expression of a wild-type counterpart to a SNP or indel is the level of expression of the wild-type in cells of a subject, e.g., a human patient, not afflicted with a cancer or with the condition under analysis. An “over-expression” or “significantly higher level of expression” of a SNP or indel refers to an expression level in a test sample that is greater than the control of the assay employed to assess expression, and is in some embodiments at least 1.1 times, and in some embodiments may be 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression level of the wild-type counterpart in a control sample (e.g., sample from a healthy subject not having the SNP or indel associated disease) and, in some embodiments, the average expression level of the wild-type in several control samples. A “significantly lower level of expression” of a SNP or indel refers to an expression level in a test sample that is at least 1.1 times, and in some embodiments may be 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the SNP or indel in a patient undergoing cancer therapy or in remission.

The term “primer” or “nucleic acid primer” or “nucleic acid primer sequence” or “oligomer” or “probe” includes single-stranded nucleotides, mononucleotides and oligonucleotides that, typically, are between about 1 to about 100 bases, or alternatively between about 17 to 30 bases, or alternatively 20 or more bases, and are designed to hybridize with a corresponding template nucleic acid or target sequence, e.g., SNPs or indel of interest. Primer molecules can be complementary to either the sense or the anti-sense strand of a template nucleic acid and flank a nucleic acid region, e.g., SNP of interest. The primers can be composed of DNA and/or RNA and/or synthetic nucleotide analogs. Additional primer modifications may comprise chain termination, acrydite-modifications, and the like. Primers can be either synthesized by one skilled in the art, or derived from appropriate biological preparations. For purposes of detection of the target molecule, primers can be specifically designed to be labeled, as described herein.

In some embodiments, indels do not have any specific sequence, hindering conventional genotyping approaches based on hybridization or primer extension because a different primer would be required for every possible indel permutation.

In such embodiments, the variable nature of the indels may be overcome by the lack of binding of a primer to the variable region. For example, the probe may be complementary to the wild-type sequence, and fails to hybridize, under stringent conditions, to the mutant type. As in other embodiments of the methods of the invention, the indel probe may be blocked for extension by a 3′ modification. Many such modifications are known to those skilled in the art, including but not limited to phosphorylation and dideoxynucleotide terminators. The 5′ end of the indel probe overlaps or approaches the constant domain of the target DNA. A second “chaser” primer may be hybridized to the constant domain, binding to both wild-type and mutant DNA. A ligation step may be performed to heal the single strand nick between the chaser and the indel probe on the wild-type template, preventing any further extension. However, the chaser probe may be extended to full length on the mutant template either into or completely through the variable domain.

In other embodiments, the indel primer may not be blocked, rather in the next step the indel primer is fully extended along the wild-type template. The wild-type template is now susceptible to restriction enzyme digestion, whereas the mutant remains single stranded. After digestion and washing, the solid support may be substantially enriched for the mutant type, still present as single strands. As needed, the complementary strand for the mutant may be synthesized using the original PCR primer.

The term “response to cancer therapy” or “outcome of cancer therapy” relates to any response of the hyperproliferative disorder (e.g., cancer) to a cancer therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection for solid cancers. Responses may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to copy number, level of expression, level of activity, etc. of one or more SNPs or indels described herein that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom the measurement values are known. In certain embodiments, the same doses of cancer therapeutic agents are administered to each subject. In related embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Outcomes can also be measured in terms of a “hazard ratio” (the ratio of death rates for one patient group to another; provides likelihood of death at a certain time point), “overall survival” (OS), and/or “progression free survival.” In certain embodiments, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, and greater, or any range in between, with respect to a relevant outcome (e.g., accuracy, sensitivity, specificity, 5-year survival, 10-year survival, metastasis-free survival, stage prediction, and the like). In a further embodiment, a significant increase in risk is at least about 50%. Thus, the present invention further provides methods for making a treatment decision for a cancer patient, comprising carrying out the methods for prognosing a cancer patient according to the different aspects and embodiments of the present invention, and then weighing the results in light of other known clinical and pathological risk factors, in determining a course of treatment for the cancer patient. For example, a cancer patient that is shown by the methods of the invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation. In addition, it will be understood that the cancer therapy responses can be predicted by the methods described herein according to enhanced sensitivity and/or specificity criteria. For example, sensitivity and/or specificity can be at least 0.80, 0.81, 0.2, 0.83, 0.84, 0.85, 0.86, 0.87, 0.88, 0.89, 0.90, 0.91, 0.92, 0.93, 0.94, 0.95, 0.96, 0.97, 0.98, 0.99 or greater, any range in between, or any combination for each of sensitivity and specificity.

The term “sample” used for detecting or determining the presence or level of at least one SNP or indel is typically whole blood, plasma, serum, saliva, urine, stool (e.g., feces), tears, and any other bodily fluid (e.g., as described above under the definition of “body fluids”), or a tissue sample (e.g., biopsy) or surgical resection tissue. In certain instances, the method of the present invention further comprises obtaining the sample from the individual prior to detecting or determining the presence or level of at least one SNP or indel in the sample. In some embodiments, the sample is received from a doctor, hospital, physician, or health care provider. In some embodiments, the sample is received by a testing laboratory or agency and tested by a user of the kits and methods of the present invention to provide a quantitative or qualitative assessment or result of the SNP or indel levels in a sample. The results may be provided to the requesting respective doctor, hospital, physician, or health care provider via paper report, in electronic form, or as part of an encrypted medical database.

The term “sensitize” means to alter cancer cells or tumor cells in a way that allows for more effective treatment of the associated cancer with a cancer therapy (e.g., chemotherapeutic or radiation therapy. In some embodiments, normal cells are not affected to an extent that causes the normal cells to be unduly injured by the cancer therapy (e.g., chemotherapy or radiation therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “subject” refers in one embodiment to an animal in need of therapy for, or susceptible to, a condition or its sequelae. The subject can include dogs, cats, pigs, cows, sheep, goats, horses, rats, mice, monkeys, and humans.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “target nucleic acid” or “template” includes any nucleic acid, e.g., SNP, intended to be detected, copied, and the like in, e.g., a polymerase amplification reaction, such as PCR.

The term “targeting polynucleotide sequence” as used herein, refers to a polynucleotide sequence which is comprised of nucleotides which are complementary to a target nucleotide sequence such that the sequence is of sufficient length and complementarity with the target sequence to form a duplex which has sufficient stability for the purpose intended. In certain methods of the invention, the variable nature of indels is overcome by the lack of binding of a targeting polynucleotide sequence to the variable region.

Overview

Compositions and methods are provided for detecting, diagnosing and prognosing an individual having or suspected of having cancer. Said compositions are used to enrich SNPs and indels significantly and abundantly over wild-type levels for ease and improved detection. In some embodiments, the invention considers reducing the gap between very rare variants and abundant DNA, such as reducing the ratio of mutant to wild-type DNA from 1:10⁶ to 1:10⁴, for ease and improved detection. Moreover, such compositions and methods can be used for the early diagnosis of or prognosticating recurrence or metastasis, which is essential to assure the best treatment regimen and outcome.

In one embodiment of the invention, one genotype of DNA is marked for selective degradation, usually the wild-type, by transforming the difference in genetic information into a physical susceptibility to enzyme degradation.

In one embodiment of the invention, the wild-type is degraded by a single-strand specific nuclease, resulting in an enrichment of mutant-type DNA. Advantages of enrichment include boosting the specificities of existing genotyping assays. For example, certain common techniques have lower limits of specificity of 1 mutant per 10,000 wild-type molecules. However, for certain liquid biopsies such as cfDNA, 1:10⁴ actually limits the assay to later stages of cancer. Performing enrichment could push specificities to 1:10⁵ or higher potentially allowing earlier identification of stage I and II cancers.

In a second embodiment of the invention, the wild-type DNA is either degraded or not, however a detectable probe for double-stranded DNA is added, preferably a fluorescent DNA intercalating dye such as YoYo-1. The amount or ratio of mutant-to-wild-type DNA is indicated by the intensity of fluorescence.

As an example, in one method of the invention the template DNA is incorporated into hydrogel microspheres such that each sphere or particle contains >10⁵ clones of only one genotype. In this case, after carrying out the methods of the invention described above, those particles containing only mutant DNA will fluoresce brightly after staining, whereas particles with wild-type DNA will fluoresce dimly or not at all. Potential advantages of this genotyping method over others, such as fluorescence hybridization, for such hydrogel particles is that enzymatic extension and exonuclease reactions are less susceptible to confined environment effects within the pores of a hydrogel.

While the invention for genotyping and enrichment is not limited to DNA bound to a solid support such as a hydrogel particle, or a microbead, or a spotted surface, or any other solid supports known by those practiced in the art, for the purpose of this embodiment of the invention, the solid support facilitated manipulation of the DNA, such as collecting the DNA after washing by centrifugation. In other embodiments of the invention, such as when the contents of each droplet contain clonal DNA from an emulsion PCR reaction at limiting dilution, the solid support also serves to permanently co-localize the DNA within the droplet. In the case of emulsion PCR, the original individual molecules within the sample are transformed into particles containing millions of identical copies of the original sequence.

Methods of the invention described thus far have pertained to known mutations, typically SNPs, with unique mutant sequences that can be targeted by hybridization and extension. However, the invention is not limited in this regard. Rather the invention also envisions genotyping and enrichment of an equally important class of variable mutations, commonly called “indels”, short for small insertions, deletions, and replacements. Indels do not have any specific sequence, hindering conventional genotyping approaches based on hybridization or primer extension because a different probe would be required for every possible indel permutation, an impractical prospect at best. In certain methods of the invention, the variable nature of indels is overcome by the lack of binding of a probe to the variable region.

Compositions

Compositions of the invention can include synthetic nucleic acid primers or probes modified with chain termination or acrydite for amplifying or enriching a SNP in a sample. Some examples include, but not limited to, the nucleic acids set forth in Tables 1 and 3.

The synthetic nucleic acid primers or probes can include polynucleotides comprising the entire or partial sequence of the nucleotide sequence encompassing the SNP or indel, or the complement of such sequences. As used herein, “polynucleotide” means a polymer of nucleic acids or nucleotides that, unless otherwise limited, encompasses naturally occurring bases (i.e., adenine, guanine, cytosine, thymine and uracil) or known base analogues having the essential nature of naturally occurring nucleotides in that they hybridize to single-stranded nucleic acid molecules in a manner similar to naturally occurring nucleotides. Although it may comprise any type of nucleotide units, the term generally applies to nucleic acid polymers of ribonucleotides (“RNA”) or deoxyribonucleotides (“DNA”). The term includes single-stranded nucleic acid polymers, double-stranded nucleic acid polymers, and RNA and DNA made from nucleotide or nucleoside analogues that can be identified by their nucleic acid sequences, which are generally presented in the 5′ to 3′ direction (as the coding strand), where the 5′ and 3′ indicate the linkages formed between the 5′ hydroxyl group of one nucleotide and the 3′-hydroxyl group of the next nucleotide. For a coding strand presented in the 5′-3′ direction, its complement (or non-coding strand) is the strand that hybridizes to that sequence according to Watson-Crick base pairing. Thus, as used herein, the complement of a nucleic acid is the same as the “reverse complement” and describes the nucleic acid that in its natural form, would be based paired with the nucleic acid in question.

As used herein, a “nucleic acid,” “nucleotide” or “nucleic acid residue” are used interchangeably to mean a nucleic acid that is incorporated into a molecule such as a gene or other polynucleotide. As noted above, the nucleic acid may be a naturally occurring nucleic acid and, unless otherwise limited, may encompass known analogues of natural nucleic acids that can function in a similar manner as naturally occurring nucleic acids. Examples of nucleic acids include any of the known base analogues of DNA and RNA such as, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The primers and probes can include not only the entire SNP or mutant sequence but also fragments and/or variants thereof. As used herein, “fragment” or “fragments” means a portion of the nucleic acid sequence of the mutated sequence. Polynucleotides that are fragments of a nucleic acid sequence generally comprise at least about 10, 15, 20, 50, 75, or 100, contiguous nucleotides, or up to the number of nucleotides present in a full-length.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

As used herein, “variant” or “variants” means substantially similar sequences. Generally, variants of a particular primer have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity (preferably over the full length) as determined by sequence alignment programs.

One of skill in the art understands that variants can be constructed via modifications to either the polynucleotide sequence of the primer, oligomer, or probe and can include substitutions, insertions (e.g., adding no more than ten nucleotides or amino acid) and deletions (e.g., deleting no more than ten nucleotides or amino acids). Methods of mutating and altering nucleic acid sequences, as well as DNA shuffling, are well known in the art. See, e.g., Crameri et al. (1997) Nature Biotech. 15:436-438; Crameri et al. (1998) Nature 391:288-291; Kunkel (1985) Proc. Natl. Acad. Sci. USA 82:488-492; Kunkel et al. (1987) Methods in Enzymol. 154:367-382; Moore et al. (1997) J. Mol. Biol. 272:336-347; Stemmer (1994) Proc. Natl. Acad. Sci. USA 91:10747-10751; Stemmer (1994) Nature 370:389-391; Zhang et al. (1997) Proc. Natl. Acad. Sci. USA 94:4504-4509; and Techniques in Molecular Biology (Walker & Gaastra eds., MacMillan Publishing Co. 1983) and the references cited therein; as well as U.S. Pat. Nos. 4,873,192; 5,605,793 and 5,837,458.

Methods of aligning sequences for comparison are well known in the art. Thus, the determination of percent sequence identity between any two sequences can be accomplished using a mathematical algorithm. Non-limiting examples of such mathematical algorithms are the algorithm of Myers & Miller (1988) CABIOS 4:11-17; the local alignment algorithm of Smith et al. (1981) Adv. Appl. Math. 2:482; the global alignment algorithm of Needleman & Wunsch (1970) J. Mol. Biol. 48:443-453; the search-for-local alignment method of Pearson & Lipman (1988) Proc. Natl. Acad. Sci. USA 85:2444-2448; the algorithm of Karlin & Altschul (1990) Proc. Natl. Acad. Sci. USA 87:2264, modified as in Karlin & Altschul (1993) Proc. Natl. Acad. Sci. USA 90:5873-5877.

Kits

Compositions of the invention can include kits for detecting, diagnosing and prognosing an individual having or suspected of having cancer. As used herein, “kit” or “kits” means any manufacture (e.g., a package or a container) including at least one reagent useful for enriching a mutant gene in a sample, such as a nucleic acid primer or probe for specifically detecting the SNP or indel. In some embodiments, a plurality of reagents is used. As used herein, “plurality” means two or more probes or primers and includes a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more or any range inclusive (e.g., 2-10 primers or probes), wherein each primer or probe of the combination selectively binds to a specifically intended target biomolecule, e.g., SNP or indel.

In other embodiments, primer (e.g., oligonucleotide) sequences are useful for detecting or analyzing gene expression of mutant genes, e.g., SNPs or indels. In one embodiment, the present invention features an oligonucleotide or primer pairs selected from the group consisting of oligonucleotides shown in Tables 1 and 3. In still another embodiment, the oligonucleotides of the invention are at least 70%, 71%, 72%, 73%, 74%, 75%, 76%, 77%, 78%, 79%, 80%, 81%, 82%, 83%, 84%, 85%, 86%, 87%, 88%, 89%, 90%/a, 91%, 92%, 93%, 94%, 95%, 96%, 97%, 98%, 99% or more identical to the nucleotide sequences set forth in oligonucleotides shown in Tables 1 and 3. In yet another embodiment, the oligonucleotides of oligonucleotides shown in Tables 1 and 3 are at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 54, 56, 58, 60, 62, 64, 66, 68, 70, 72, 74, 76, 78, 80 or more nucleotides in length. A skilled artisan will appreciate that certain elements of the useful oligonucleotides described herein (e.g., restriction enzyme sites, cloning sites, overlapping linker sites, shortened, lengthened, and/or modified in sequence for suitable annealing temperature design, etc.) can readily be altered. It will be appreciated that the nucleic acid sequences of the present invention need not consist only of the sequence which is complementary to the targeted locus of genetic variation. Thus, the nucleic acid sequences of the present invention can contain in addition, nucleotide sequences or other moieties, e.g., chain termination or acrvdite, which are suitable for the purposes for which the nucleic acid sequences are used.

In one embodiment, the invention provides a combination of one or more oligonucleotides of the present invention (e.g., useful as probes). In yet another embodiment, the invention provides a set of oligonucleotides, also referred to herein as “primer pairs” and “nucleic acid primer sequences,” selected from the group consisting of two or more of the oligonucleotides of the present invention. In still another embodiment, the invention provides oligonucleotides which are able to amplify a SNP having a nucleotide sequence selected from the group consisting of oligonucleotides shown in Tables 1 and 3, or a complement thereof.

In another embodiment, the oligonucleotides of the present invention comprise a label for detection. Such labels can be, e.g., radioactive labels which can be incorporated by known methods (e.g., nick translation or kinasing), radioactive isotopes, biotin, fluorescent groups, chemiluminescent groups (e.g., dioxetanes, particularly triggered dioxetanes), digoxigenin, enzymes, antibodies, luminescent agents, precipitating agents, dyes, combinations thereof, and the like.

In another aspect, the kit comprises a set of primers selected from the group consisting of the oligonucleotides of the present invention. The primers in such kits can be labeled or unlabeled. The kit can also include additional reagents such as reagents for performing an amplification (e.g., PCR) reaction, a reverse transcriptase for conversion of RNA to cDNA for amplification, DNA polymerases, dNTP and ddNTP feedstocks. Kits of the present invention can also include instructions for use.

Methods of synthesizing polynucleotides are well known in the art, such as cloning and digestion of the appropriate sequences, as well as direct chemical synthesis (e.g., ink-jet deposition and electrochemical synthesis). Methods of cloning polynucleotides are described, for example, in Copeland et al. (2001) Nat. Rev. Genet. 2:769-779; Current Protocols in Molecular Biology (Ausubel et al. eds., John Wiley & Sons 1995); Molecular Cloning: A Laboratory Manual, 3^{rd} ed. (Sambrook & Russell eds., Cold Spring Harbor Press 2001); and PCR Cloning Protocols, 2^{nd} ed. (Chen & Janes eds., Humana Press 2002). Methods of direct chemical synthesis of polynucleotides include, but are not limited to, the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al. (1991) Science 251:767-773; Pease et al. (1994) Proc. Natl. Acad. Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad. Sci. USA 76:1760-1764; as well as EP Patent No. 1721908; Int'l Patent Application Publication Nos. WO 2004/022770 and WO 2005/082923; US Patent Application Publication Nos. 2009/0062521 and 2011/0092685; and U.S. Pat. Nos. 6,521,427; 6,818,395; 7,521,178 and 7,910,726.

The kits can be promoted, distributed or sold as units for performing the methods described below. Additionally, the kits can contain a package insert describing the kit and methods for its use. For example, the insert can include instructions for correlating the level of SNP or indel expression measured with a subject's likelihood of having developed cancer or the likely prognosis of a subject already diagnosed with cancer.

The kits therefore can be for detecting, diagnosing and prognosing a cancer with SNPs or indels at the nucleic acid level. Such kits are compatible with both manual and automated nucleic acid detection techniques using fluorescence. These kits can include a plurality of probes or reagents, for example, from two to thirty nucleic acid probes or reagents that specifically bind to the amplicons. Alternatively, the kits can contain at least two probes, at least three probes, at least four probes, at least five probes, at least six probes, at least seven probes, at least eight probes, at least nine probes, at least ten probes, at least eleven probes, at least twelve probes, at least thirteen probes, at least fourteen probes, at least fifteen probes, at least sixteen probes, at least seventeen probes, at least eighteen probes, at least nineteen probes, at least twenty probes, at least twenty-five probes, or at least thirty probes.

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, RNA preparations from tissues or cell lines, and the like, known to be either positive or negative for the presence of the SNP or indel. The design and use of controls is standard and well within the routine capabilities of one of skill in the art.

Methods

Methods of Enriching Mutant-Type Nucleic Acids

One aspect of the invention relates to a method comprising: providing a nucleic acid target, the nucleic acid target comprising: a locus of genetic variation, and a conserved region on the 3′-side of the locus of genetic variation; dissociating any associated strands within the nucleic acid target; hybridizing an oligomer within the conserved region adjacent to the 3′-side of the locus of genetic variation; extending the oligomer with a first chain terminating nucleotide (e.g., mononucleotide) that is sequence-specific to one genotype of the nucleic acid target, the extension reaction yielding: a chain-terminated product if the sequence is matching, or no affect if the sequence is mismatching; replacing the first chain terminating nucleotide (e.g., mononucleotide) with a second mixture of extensible nucleotides (e.g., mononucleotides); extending the oligomer with the second mixture of extensible nucleotides (e.g., mononucleotides), yielding: no affect if the oligomer was chain-terminated, yielding a terminated oligomer, or an extended oligomer product if the oligomer was extensible, yielding an extended oligomer. As used herein, “first chain terminating nucleotide” is used interchangeably with “first chain terminating mononucleotide”. A common acronym for “first chain terminating nucleotide” or “first chain terminating mononucleotide” is ddNTP for dideoxynucleotide, and yet more specifically ddATP, ddTTP, ddGTP, and ddCTP. In some embodiments, the melting temperature of the complex between the nucleic acid target and the extended oligomer is higher than the melting temperature of the complex between the nucleic acid target and the terminated oligomer. In some embodiments, the melting temperature of the complex between the nucleic acid target and the extended oligomer is at least five degrees Celsius different than the melting temperature of the complex between the nucleic acid target and the terminated oligomer. In some embodiments, the temperature is poised below the melting temperature of the complex between the nucleic acid target and the extended oligomer, yielding associated double-stranded DNA, and above the melting temperature of the complex between the nucleic acid target and the terminated oligomer, yielding dissociated single-stranded DNA. In some embodiments, the single-stranded DNA is degraded by enzymatic digestion, but the double-stranded DNA remains intact. In some embodiments, the double-stranded DNA is detected with a DNA recognition agent or DNA recognition system. In some embodiments, the DNA recognition agent is a fluorescent intercalating dye. In some embodiments, the nucleic acid target is tethered to a solid support. In some embodiments, the nucleic acid target is tethered to a microsphere, microbead, or nanoparticle. In some embodiments, the solid support is a hydrogel. In some embodiments, the hydrogel is a microparticle. In some embodiments the microparticle is streptavidin-coaded microparticles, such as DynaBeads described supra.

Another aspect of the invention relates to a method comprising: providing a nucleic acid target, the nucleic acid target comprising: a locus of genetic variation, and a conserved region on the 3′-side of the locus of genetic variation; dissociating any associated strands within the nucleic acid target; hybridizing a first oligomer overlapping the locus of genetic variation, the first oligomer comprising: a recognition domain for the wild-type genetic sequence, and a chain-terminating nucleotide on the 3′-side, and yielding a double-stranded DNA complex with the nucleic acid target in the presence of the wild-type sequence, but not for genetic variants; hybridizing a second oligomer substantially within the conserved region and immediately adjacent to the first oligomer, and yielding a double-stranded DNA complex with the nucleic acid target for both the wild-type sequence and genetic variants; ligating the first and second oligomers, yielding a double-stranded complex between the nucleic acid target and a chain terminated combination of the first and second oligomers for the wild-type nucleic acid target, yielding terminated oligomer, or an extensible second oligomer for genetic variants, yielding extensible oligomer; extending the oligomers, yielding: no affect for terminated oligomers, or an extended oligomer product for extensible oligomers. In some embodiments, the nucleic acid targets are tethered to a solid support. In some embodiments, the nucleic acid target is tethered to a microsphere, microbead, or nanoparticle. In some embodiments, the solid support is a hydrogel. In some embodiments, the hydrogel is a microparticle. In some embodiments the microparticle is streptavidin-coaded microparticles, such as DynaBeads described supra. In some embodiments, the extended oligomers products arising from nucleic acid targets with mutations are selectively released from the solid support by cleavage of the double-stranded DNA. In some embodiments, the double-stranded DNA is cleaved by restriction enzyme digestion.

Another aspect of the invention relates to a method comprising: providing a nucleic acid target, the nucleic acid target comprising a locus of genetic variation; dissociating any associated strands within the nucleic acid target; hybridizing an oligomer overlapping the locus of genetic variation, and yielding a double-stranded DNA complex with the nucleic acid target in the presence of the wild-type sequence, but not for genetic variants; extending the oligomer, yielding: an extended oligomer product for oligomers bound to the wild-type nucleic acid target wherein the oligomer product and the wild-type nucleic acid target form a double-stranded complex, or no product for the mutant-type nucleic acid target. In some embodiments, the nucleic acid targets are tethered to a solid support. In some embodiments, the hydrogel is a microparticle. In some embodiments, the solid support is a hydrogel. In some embodiments, the hydrogel is a microparticle. In some embodiments the microparticle is streptavidin-coaded microparticles, such as DynaBeads described supra. In some embodiments, the extended oligomers products arising from nucleic acid targets with mutations are selectively released from the solid support by cleavage of the double-stranded DNA. In some embodiments, the double-stranded DNA is cleaved by restriction enzyme digestion.

Methods of Detecting and/or Diagnosing Cancers

Methods of the invention include detecting and/or diagnosing a cancer in an individual having or suspected of having a cancer using the aforementioned methods of enriching mutant-type nucleic acids. The method can include determining the expression levels of SNPs or indels in a sample from an individual having or suspected of having a cancer. Said method enriches the sample with the mutant genotype. Advantages of enrichment include boosting the specificities of existing genotyping assays. For example, certain common techniques have lower limits of specificity of 1 mutant per 10,000 wild-type molecules. However, for certain liquid biopsies such as cfDNA, 1:10⁴ actually limits the assay to later stages of cancer. Performing enrichment could push specificities to 1:10⁵ or higher potentially allowing earlier identification of stage I and II cancers.

The methods generally begin by collecting a sample from an individual having or suspected of having a cancer. As used herein, “sample” means any collection of cells, tissues, organs or bodily fluids in which expression of a locus of genetic variation can be detected. Examples of such samples include, but are not limited to, biopsy specimens of cells, tissues or organs, bodily fluids and smears.

Biopsy specimens can be obtained by a variety of techniques including, but not limited to, scraping or swabbing an area, using a needle to aspirate cells or bodily fluids, or removing a tissue sample. Methods for collecting various body samples/biopsy specimens are well known in the art.

Fixative and staining solutions can be applied to, for example, cells or tissues for preserving them and for facilitating examination. Body samples, particularly thymus tissue samples, can be transferred to a glass slide for viewing under magnification. In one embodiment, the body sample is a formalin-fixed, paraffin-embedded tissue sample, particularly a primary tumor sample.

When the sample is a bodily fluid, it can include, but is not limited to, blood, lymph, urine, saliva, aspirates or any other bodily secretion or derivative thereof. When the sample is blood, it can include whole blood, plasma, serum or any derivative of blood.

Methods of Prognosing Cancers

Methods of the invention include prognosing the likelihood of metastasis in an individual having a cancer. The methods include detecting the expression of SNP or indel in a sample from an individual having a cancer. Altered expression levels of a SNP or indel can be used to indicate cancer prognosis (i.e., poor or good prognosis). As such, altered expression of a particular SNP or indel permits the differentiation of individuals having a cancer that are likely to experience disease recurrence and/or metastasis (i.e., poor prognosis) from those who are more likely to remain cancer free (i.e., good prognosis).

As used herein, “prognose,” “prognoses,” “prognosis” and “prognosing” means predictions about or predicting a likely course or outcome of a disease or disease progression, particularly with respect to a likelihood of, for example, disease remission, disease relapse, tumor recurrence, metastasis and death (i.e., the outlook for chances of survival). As used herein, “good prognosis” or “favorable prognosis” means a likelihood that an individual having cancer will remain disease-free (i.e., cancer-free). As used herein, “poor prognosis” means a likelihood of a relapse or recurrence of the underlying cancer or tumor, metastasis or death. Individuals classified as having a good prognosis remain free of the underlying cancer or tumor. Conversely, individuals classified as having a bad prognosis experience disease relapse, tumor recurrence, metastasis or death.

Additional criteria for evaluating the response to anti-cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

One of skill in the art is familiar with the time frame(s) for assessing prognosis and outcome. Examples of such time frames include, but are not limited to, less than one year, about one, two, three, four, five, six, seven, eight, nine, ten, fifteen, twenty or more years. With respect to cancer, the relevant time for assessing prognosis or disease-free survival time often begins with the surgical removal of the tumor or suppression, mitigation or inhibition of tumor growth. Thus, for example, a good prognosis can be a likelihood that the individual having cancer will remain free of the underlying cancer or tumor for a period of at least about five, more particularly, a period of at least about ten years. In contrast, for example, a bad prognosis can be a likelihood that the individual having cancer experiences disease relapse, tumor recurrence, metastasis or death within a period of less than about five years, more particularly a period of less than about ten years.

The expression levels of at least one SNP or indel in a tumor sample can be indicative of a poor cancer prognosis and thereby used to identify individuals who are more likely to suffer a recurrence of the underlying cancer. The therefore methods involve detecting the expression levels of at least one SNP or indel r in a tumor sample that is indicative of early stage disease.

In some embodiments, overexpression of a SNP or indel of interest in a sample can be indicative of a poor cancer prognosis. As used herein, “indicative of a poor prognosis” is intended that altered expression of particular SNP or indel is associated with an increased likelihood of relapse or recurrence of the underlying cancer or tumor, metastasis or death. For example, “indicative of a poor prognosis” may refer to an increased likelihood of relapse or recurrence of the underlying cancer or tumor, metastasis, or death within ten years, such as five years. In other aspects of the invention, the absence of overexpression of a SNP or indel of interest is indicative of a good prognosis. As used herein, “indicative of a good prognosis” refers to an increased likelihood that the patient will remain cancer free. In some embodiments, “indicative of a good prognosis” refers to an increased likelihood that the patient will remain cancer-free for ten years, such as five years.

In certain embodiments, the methods of the invention implement a computer program and computer system. For example, a computer program can be used to perform the algorithms described herein. A computer system can also store and manipulate data generated by the methods of the present invention which comprises a plurality of SNP or indel signal changes/profiles which can be used by a computer system in implementing the methods of this invention. In certain embodiments, a computer system receives SNP or indel enrichment data; (ii) stores the data; and (iii) compares the data in any number of ways described herein (e.g., analysis relative to appropriate controls) to determine the state of informative SNPs or indels from cancerous or pre-cancerous tissue. In other embodiments, a computer system (i) compares the determined SNP or indel level to a threshold value; and (ii) outputs an indication of whether said SNP or indel level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of SNP or indel enrichment data. Such stored profiles can be accessed and used to perform comparisons of interest at a later point in time. For example, SNP or indel enrichment profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Methods of Treating Cancers

The compositions, kits and detection, diagnosing and prognosing methods described above can be used to assist in selecting appropriate treatment regimen and to identify individuals that would benefit from more aggressive therapy.

As noted above, approaches to the treating cancers include surgery, immunotherapy, chemotherapy, radiation therapy, a combination of chemotherapy and radiation therapy, or biological therapy. Chemotherapeutics that have been used in the treatment of carcinomas include, but are not limited to, doxorubicin (Adriamycin), cisplatin, ifosfamide, and corticosteroids (prednisone). Often, these agents are given in combination to increase their effectiveness. Combinations used to treat cancer include the combination of cisplatin, doxorubicin, etoposide and cyclophosphamide, as well as the combination of cisplatin, doxorubicin, cyclophosphamide and vincristine.

The methods described above therefore find particular use in selecting appropriate treatment for early-stage cancer patients. The majority of individuals having cancer diagnosed at an early-stage of the disease enjoy long-term survival following surgery and/or radiation therapy without further adjuvant therapy. However, a significant percentage of these individuals will suffer disease recurrence or death, leading to clinical recommendations that some or all early-stage cancer patients should receive adjuvant therapy (e.g., chemotherapy). The methods of the present invention can identify this high-risk, poor prognosis population of individuals having early-stage cancer and thereby can be used to determine which ones would benefit from continued and/or more aggressive therapy and close monitoring following treatment. For example, individuals having early-stage cancer and assessed as having a poor prognosis by the methods disclosed herein may be selected for more aggressive adjuvant therapy, such as chemotherapy, following surgery and/or radiation treatment. In particular embodiments, the methods of the present invention may be used in conjunction with standard procedures and treatments to permit physicians to make more informed cancer treatment decisions.

### EXAMPLES

The invention will be more fully understood upon consideration of the following non-limiting examples, which are offered for purposes of illustration, not limitation.

### Example 1: A Primer Extension Assay for Detection of EGFR L858R Mutation Associated with Lung Cancer

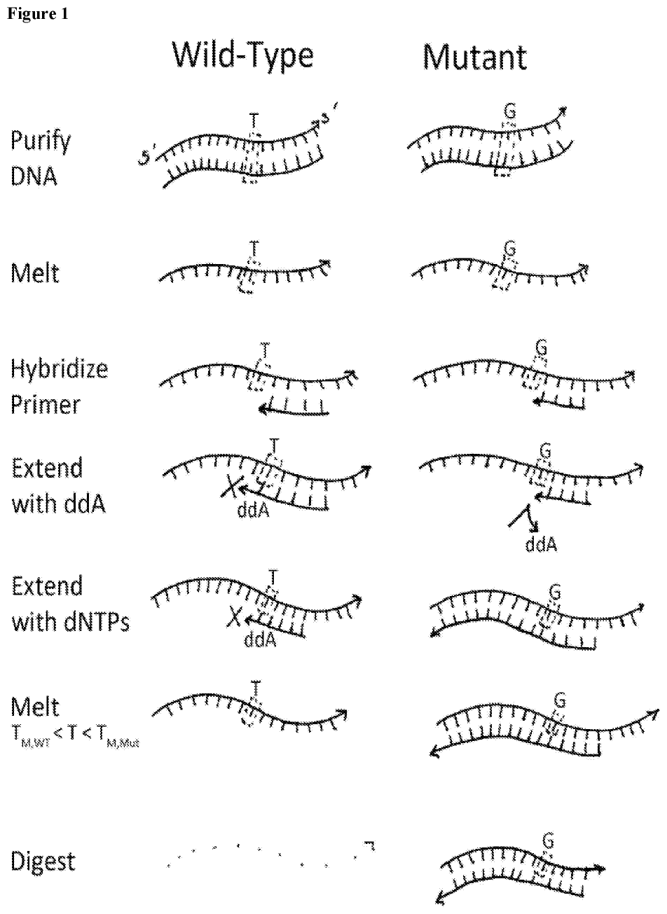

A primer extension assay incorporates a chain-terminating and base-pair specific moiety, preferably a dideoxynucleotide, at the site of a wild-type base within the locus of a targeted mutation (FIG. 1). Any mutant-type DNA present in the same reaction will not have a chain terminated primer. For example, for the L858R mutation in the human EGFR gene that is associated with lung cancer, the DNA mutation is a threonine to guanosine at position c.2573. Therefore, a primer hybridized immediately adjacent to position c.2573 on the 3′-side of the template will be extended one base by a DNA polymerase in the presence of dideoxyadenosine (ddA) nucleotides. However, the reaction mix lacks dideoxycytosine (ddC) and therefore a primer bound to mutant DNA is not extended, nor terminated. At this stage the genetic difference is transformed into a difference in extensibility of the bound primers. In the next step, the dideoxynucleotides are washed out and replaced with standard deoxynucleotides (A, T, G, and C, also called dNTPs) for a second round of extension. The primer bound to wild-type DNA is unable to extend further due to the chain termination. The primer bound to the mutant-type can extend to the limit of the DNA template, which in the preferred embodiment is sufficiently long to substantially increase the melting temperature (T_{M}) of the complex. At the second step, the original difference in sequence is transformed into a difference in T_{M}.

In the third step the temperature is raised between the T_{MS} of the terminated short (low T_{M}) wild-type strand and the long (high T_{M}) mutant strand. The original wild-type strand unbinds from the terminated primer, yielding a single-stranded piece of DNA. The mutant complex remains stable as double stranded DNA (dsDNA). Single-stranded DNA is susceptible to degradation by exonuclease activity, hence the original sequence difference is transformed into a different susceptibility to exonuclease activity.

### Example 2: Genotyping

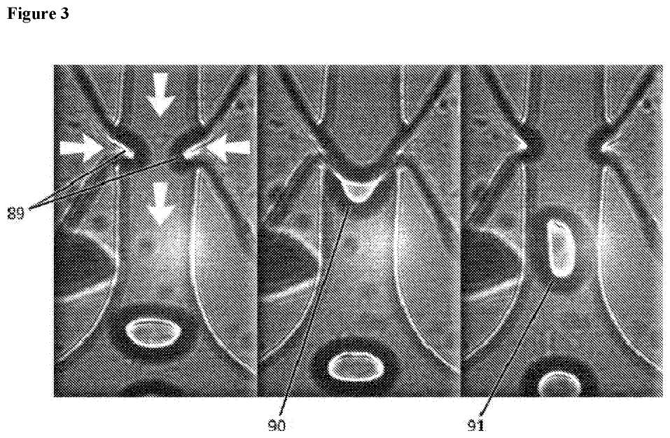

Hydrogel microparticles impregnated with DNA of either mutant or wild-type were synthesized in an emulsion as follows. First, representative regions of both genotypes of the target DNA (wild-type, SEQ ID NO: 1; mutant, SEQ ID NO:2) were synthesized (gBlocks, IDT) and then functionalized with acrydite moieties on one strand by further PCR amplification: one of the PCR primers bore a 5′-acrydite modification (SEQ ID NO:3). For sequence listing, see Table 1. The acrydite does not inhibit PCR; rather it becomes incorporated assymetrically (only on one strand) into the amplified product (amplicons). A bridge-style microdroplet generator (see FIG. 3) was loaded with acrylamide and ammonium persulfate on one line, and with target DNA amplicons and TEMED on the other (see Table 2 for final concentrations after mixing into 10 mM Tris buffer, pH 8.0). During droplet generation equal volumes from each stream were captured together and mixed within the ˜45 um diameter (˜50 pL) spherical aqueous partitions, and the resulting hydrogels took on the form of the original droplet mold. The acrydite moiety on the amplicons can participate in free-radical polymerization, hence the amplicons contained within the droplets during gel formation became covalently incorporated into the resulting hydrogel particles.

TABLE 1
Nucleic acids

| Nucleic Acid | Sequence | |
|---|---|---|
| Type | Identifier | Sequence |
| EGFR wild-type | SEQ ID | 001 TGGTGCACCG CGACCTGGCA GCCAGGAACG TACTGGTGAA |
| gene fragment | NO: 1 | AACACCGCAG |
| | | 051 CATGTCAAGA TCACAGATTT TGGGCTGGCC AAACTGCTGG |
| | | GTGCGGAAGA |
| | | 101 GAAAGAATAC CATGCAGAAG GAGGCAAAGT AAGGAGGTGG |
| | | CTTTAGGTCA |
| | | 151 G |
| EGFR mutant | SEQ ID | 001 TGGTGCACCG CGACCTGGCA GCCAGGAACG TACTGGTGAA |
| gene fragment | NO: 2 | AACACCGCAG |
| | | 051 CATGTCAAGA TCACAGATTT TGGGCGGGCC AAACTGCTGG |
| | | GTGCGGAAGA |
| | | 101 GAAAGAATAC CATGCAGAAG GAGGCAAAGT AAGGAGGTGG |
| | | CTTTAGGTCA |
| | | 151 G |
| PCR forward | SEQ ID | 5′-Acrydite-AAAACACCGCAGCATGTCAA-3′ |
| primer | NO: 3 | |
| PCR reverse | SEQ ID | 5′-CTGACCTAAAGCCACCTCCT-3′ |
| primer | NO: 4 | |
| SNP detection | SEQ ID | 5′-CCGCACCCAGCAGTTTGGCC-3′ |
| primer | NO: 5 | |

TABLE 2
Droplet composition after mixing into 10 mM Tris buffer, pH = 8.0.

| Chemical | Amount | Vendor | Part number |
|---|---|---|---|
| Acrylamide/bisacrylamide | 4% | Sigma-Aldrich | A9926 |
| (19:1) | | | |
| TEMED | 38 mM | Sigma-Aldrich | T22500 |
| Ammonium persulfate | 38 mM | Sigma-Aldrich | A3678 |
| Target DNA | 2 μg/mL | n/a | n/a |

Particles were purified from the emulsion by first aspirating the bottom oil layer and then vortexing with a volume of 1H,1H,2H,2H-perfluorooctanol (Alfa Aesar, B20156) equal to 2-3× the volume of particles, yielding a clear aqueous supernatant after centrifugation that contained the particles. The bottom fluorous layer was aspirated and the particles were washed at least twice in TE buffer (10 mM Tris, pH 8.0, 1 mM EDTA) with a final resuspension in TET buffer (TE buffer, 0.5% Tween-20).

The microparticles were resuspended and heated under vortexing (95° C., 30 min) directly from their TET storage buffer, and then twice washed twice with salt buffer (10 mM Tris, pH 8.0, 50 mM NaCl, 1.5 mM MgCl₂, and 0.5% Tween-20) to remove the free strand of DNA that originated from primers without acrydite; only the strand originating from the acrydite primer remained bound to the particle. Washing away the complementary strand exposed the bases of the bound strand to hybridization. This melting step was repeated again with the salt buffer, and the final particles were resuspended in an extension buffer (AmpliTaq Gold (ThermoFisher Scientific, N8080240) buffer (lx) and enzyme (1.25 U/25 μL), 0.9 μM “SNP detection primer” (SEQ ID NO:5), and 0.3 mM ddA (GE Healthcare, 27205101)). With only an adenosine base present in the extension buffer, the first extension reaction (95° C. hot start, 10 min; 58° C. extension, 30 min) blocked the SNP detection primer on the wild-type template from further extension, but the SNP detection primer on the mutant type remained unaffected because the reaction mixture lacked its complementary nucleotide. The particles were washed three times to dilute the ddA by ˜500×, and then resuspended in a second extension buffer (AmpliTaq Gold buffer and enzyme, and dNTPs). The hot start was performed separately to avoid dissociation of the reaction products from the first hybridization and extension. The extension was run again at 58° C. for 30 min, and then the particles were washed twice with the salt buffer. The truncated wild-type extension products were selectively melted off and washed away (72° C., 30 min). Lastly the single-stranded wild-type DNA was digested away by DNA exonuclease I (New England Biolabs, M0293S) (37° C., 30 min).

### Example 3: Genotype of Hydrogel Microparticles

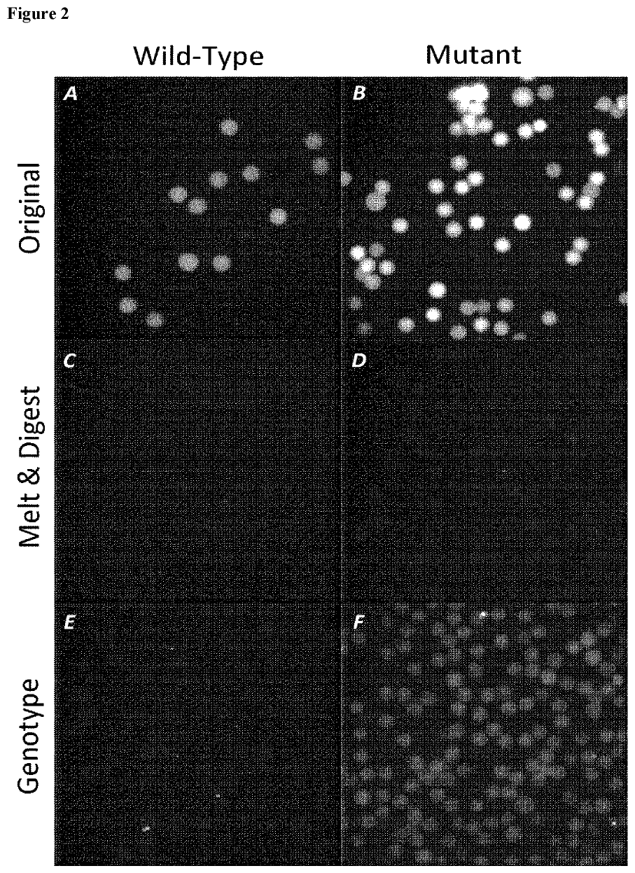

Hydrogel microparticles were genotyped by the above methods of the invention (FIG. 2). In each image, the DNA within the particles was detected by epifluorescence microscopy as described previously (see WO/2014/145555) using the double-stranded DNA intercalator YoYo-1 (ThermoFisher Scientific, Y3601). FIGS. 2A and 2B show the original DNA-impregnated particles, with bright fluorescence for both the wild-type and mutant. To demonstrate both (1) that the single-stranded DNA that melted off of the particles is mobile and can diffuse from within the particles, and (2) that the remaining bound DNA is enzyme-accessible throughout the particles, the DNA was digested by exonuclease immediately after the first melting step in the genotyping procedure. The fluorescent signal almost completely disappeared, confirming biochemical activity of the bound DNA (FIGS. 2C and 2D). FIGS. 2E and 2F show the recovery of fluorescence after the full genotyping assay in the mutant-type particles, but not in the wild-type particles that remained at background values. The positive mutant signal compared to the wild-type successfully matched the genotype, proving the concept of methods of the invention for genotyping.

### Example 4

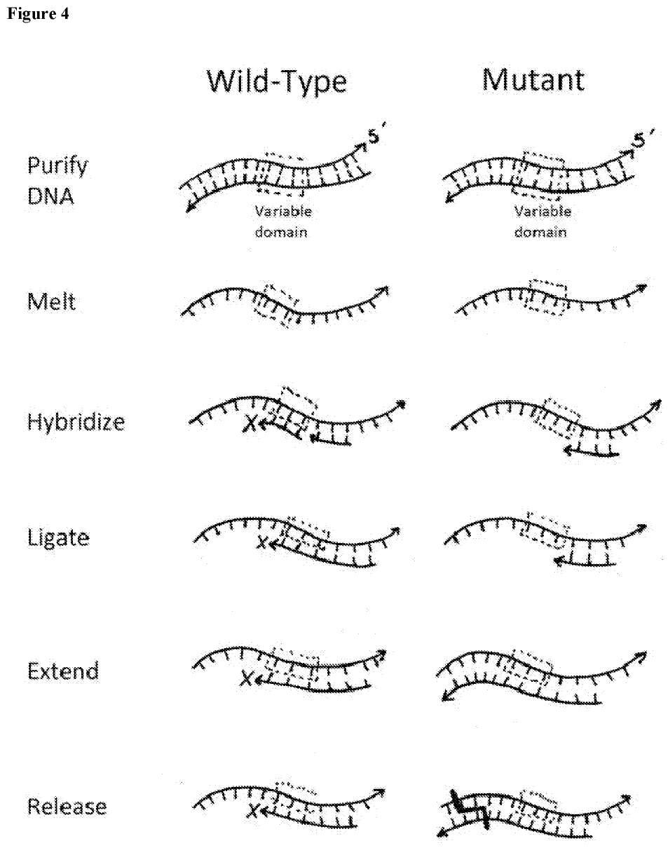

Shown in FIG. 4, one of the methods of the invention involves binding an oligonucleotide probe, the “indel probe” to the variable domain. The probe is complementary to the wild-type sequence, and fails to hybridize, under stringent conditions, to the mutant type. As in previous methods of the invention, the indel probe is blocked for extension by a 3′ modification. Many such modifications are known to those skilled in the art, including but not limited to phosphorylation and dideoxynucleotide terminators. The 5′ end of the indel probe overlaps or approaches the constant domain of the target DNA. A second “chaser” primer is hybridized to the constant domain, binding to both wild-type and mutant DNA. A ligation step is performed to heal the single strand nick between the chaser and the indel probe on the wild-type template, preventing any further extension. However, the chaser probe is extended to full length on the mutant template either into or completely through the variable domain.

The invention considers any method of distinguishing or isolating the fully extended mutant DNA from the truncated wild-type, whether a variable mutation as described above or a SNP or any other local genetic modification. In one method of the invention, shown in FIG. 4, the target DNA is purified and bound to a solid support. The template DNA also contains a restriction digestion site near the end tethered to the surface. In the preferred embodiment, the template DNA is synthesized by PCR amplification using a first primer that contains (1) a 5′-acrydite moiety, (2) a 5′-overhang, non-complementary to the target, with a custom restriction site, and (3) a 3′ region complementary to the target; and a second conventional primer that is complementary to the target. The amplicons are entrapped in hydrogel microparticles according to methods of the invention above via the acrydite linker, and the unbound complementary strand is melted off and washed away. The remaining bound strand is extended, selectively based on genotype, according to the methods described above. The two genotypes are now differentiated both by the length of the extension, i.e. T_{M}, as well as the presence of a duplex restriction site close to the mutant anchor point. The genotypes can separated or identified by either selective melting or by enzymatic release of the mutant type. The latter is preferred for variable mutations because the T_{MS} of the fully extended product and the potentially long indel+chaser construct may be similar.

### Example 5

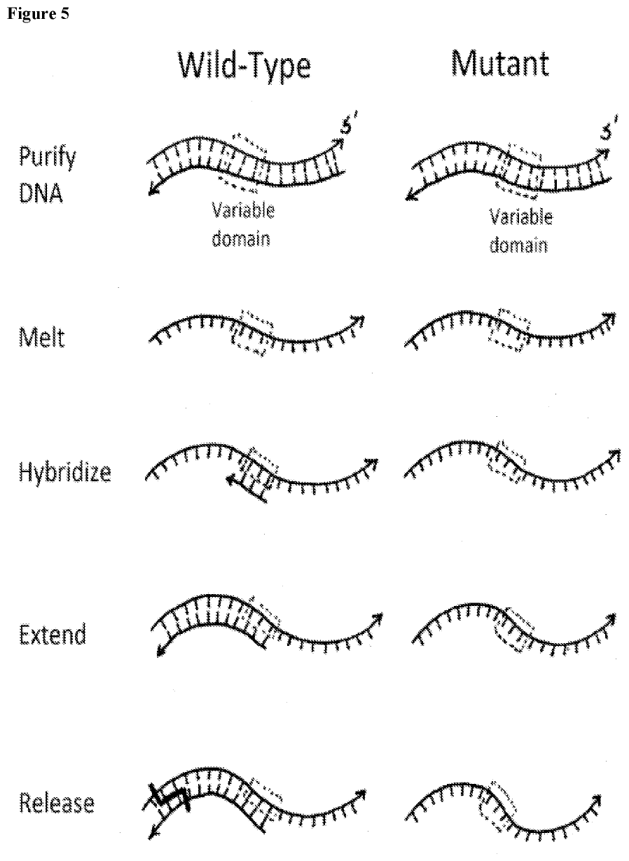

The invention has been described in the context of fully extended mutant templates alongside truncated wild-type products. The invention is not limited in this regard. The wild-type templates can also be fully extended alongside truncated mutant products, as embodied in another method of the invention for genotyping variable mutations. In this method, shown in FIG. 5, the target DNA is bound to a solid support as described above with a restriction digestion site incorporated near the tether point, and the unbound complementary strand is melted off and washed away. As above, an indel primer hybridizes specifically to the variable domain of the wild-type sequence, however in this method the indel primer is not blocked, rather in the next step the indel primer is fully extended along the wild-type template. The wild-type template is now susceptible to restriction enzyme digestion, whereas the mutant remains single stranded. After digestion and washing, the solid support is substantially enriched for the mutant type, still present as single strands. As needed, the complementary strand for the mutant may be synthesized using the original PCR primer. And, as with other methods of the invention, the genotype can be revealed by different levels of fluorescence intensity, with a brighter signal indicating the presence of the mutant.

### Example 6

Hydrogel microparticles were genotyped by above methods of the invention (FIG. 5). The starting microgels contained covalently bound DNA, prepared as follows. DNA (as shown in Table 3) was synthesized by PCR amplification of synthetic fragments (gBlock, IDT) of exon 19 from the human EGFR gene—both wild-type (SEQ ID NO: 6) and one indel-type mutant (SEQ ID NO: 7)—with a forward primer that contained an Acrydite™ moiety (SEQ ID NO: 8), a conventional reverse primer (SEQ ID NO: 9), and PCR master mix containing: TaqMan Universal Master Mix w/ no UNG (Life Technologies), plus 0.3 mM supplementary dNTPs (dNTP Mix, New England Biolabs), using the following thermal cycling: 10 min, 95° C. hot start; 35 cycles of 15 s at 94° C., 30 s at 62° C., and 10 s at 72° C.; followed by a 5 min hold at 72° C. Gel electrophoresis (E-Gel Go! 2% agarose gel, Life Technologies) revealed two distinct bands, one band for the full length wild-type sequence and another slightly shorter band for the mutant sequence that has a 12 bp deletion. DNA concentrations were 3.64 Cpg/mL and 2.52 ug/mL for the wild-type and mutant respectively, measured by quantitative fluorimetry (Qubit 2.0 with dsDNA HS Assay, Life Technologies).

TABLE 3
EGFR exon 19 fragment sequences

| Nucleic Acid | Sequence | |
|---|---|---|
| Type | Identifier | Sequence |
| EGFR exon 19 | SEQ ID | 0001 AGTGTCCCTC ACCTTCGGGG TGCATCGCTG GTAACATCCA CCCAGATCAC |
| wild-type gene | NO: 6 | 0051 TGGGCAGCAT GTGGCACCAT CTCACAATTG CCAGTTAACG TCTTCCTTCT |
| fragment | | 0101 CTCTCTGTCA TAGGGACTCT GGATCCCAGA AGGTGAGAAA GTTAAAATTC |
| | | 0151 CCGTCGCTAT CAAGGAATTA AGAGAAGCAA CATCTCCGAA AGCCAACAAG |
| | | 0201 GAAATCCTCG ATGTGAGTTT CTGCTTTGCT GTGTGGGGGT CCATGGCTCT |
| | | 0251 GAAC |
| EGFR exon 19 | SEQ ID | 0001 AGTGTCCCTC ACCTTCGGGG TGCATCGCTG GTAACATCCA CCCAGATCAC |
| mutant gene | NO: 7 | 0051 TGGGCAGCAT GTGGCACCAT CTCACAATTG CCAGTTAACG TCTTCCTTCT |
| fragment | | 0101 CTCTCTGTCA TAGGGACTCT GGATCCCAGA AGGTGAGAAA GTTAAAATTC |
| | | 0151 CCGTCGCTAT CAAGGAACCA TCTCCGAAAG CCAACAAGGA AATCCTCGAT |
| | | 0201 GTGAGTTTCT GCTTTGCTGT GTGGGGGTCC ATGGCTCTGA AC |
| EGFR exon 19 | SEQ ID | 5′-Acrydite-ATGCATGCGGATCCAGTGTCCCTCACCTTCGGGG-3′ |
| forward primer | NO: 8 | |
| EGFR exon 19 | SEQ ID | 5′-ACCCCCACACAGCAAAGCAG-3′ |
| reverse primer | NO: 9 | |
| EGFR exon 19 | SEQ ID | 5′-GGAGATGTTGCTTCTCTTAATTCCT-3′ |
| variable primer | NO: 10 | |

Hydrogel microparticles were synthesized by similar methods as above using highly uniform microdroplets emulsified in a bridge-mode droplet generator (FIG. 3). Operation of the bridge-mode droplet generator has been described in detail elsewhere (US. Publication Application No: 20160136643). In this example, both of the aqueous phases that were injected into the droplet generator contained identical mixtures of DNA amplicons and gel prepolymer. This contrasts with the examples above. In those examples the chemical initiator (ammonium persulfate) and the catalyst (TEMED) were separated into the two different flow streams. However, in this example each flow stream contained the initiator. The catalyst was introduced later, after emulsification, through an exchange of the oil. In another difference between this example and those above, microgel polymerization was performed under a chemically inert environment. Those skilled in the art will recognize that atmospheric oxygen can inhibit polymerization to deleterious effects such as reduced yield, variable gel chain lengths, and overall inconsistency. As demonstrated above, inert conditions are not a requirement of the invention. However, the preferred embodiment eliminates this potential variable. In this example, the two aqueous reagents and the oil were sparged with nitrogen for one hour prior to droplet generation.

The aqueous solution injected into the microdroplet generator contained DNA targeted at a final DNA concentration of 1 μg/mL; 10 mM Tris, pH 8.0; 1 mM EDTA; 50 mM NaCl; 6.2% acrylamide; 0.3% bisacrylamide; and 0.3% ammonium persulfate. The oil line contained HFE 7500 (3M Novec 7500 Engineered Fluid, 3M) and fluorosurfactant (RAN Biotechnologies). After generation of the microdroplets, polymerization was catalyzed by exchanging the oil with another oil—otherwise identical—with 27 mM TEMED. The emulsion containing polymerized microparticles was broken with 1H,1H,2H,2H-perfluoro-1-octanol (Sigma Aldrich) at approximately 3× volume, 300 μL, of collected emulsion, 100 μL. After centrifugation for 1 minute at 12,500 rpm, microparticles were collected from the supernatant and the fluorous subnatant was discarded. Particles were then washed twice with 1 mL of Tris, pH 8.0. Each wash consisted of adding volume, vortexing, centrifuging for 1 min at 12,500 rpm, and removing the aqueous supernatant. The stock of particles generated were suspended in Tris, pH 8.0 with 50 mM NaCl and 1.5 mM MgCl₂ (particle buffer) and stored at 8° C.

The PCR product used for particle preparation, above, contained both the full length amplicons as well as any remaining unreacted primers. In preparation for genotyping, a starting aliquot of 150 μL of stock particles per assay were first “cleaned” to remove any unextended, single-stranded acrydite primers that may have incorporated into the particles. 10 U/μL of Exonuclease I and 1× Exonuclease I buffer (New England Biolabs) was added to the particles and rotary mixed (BioShake IQ, QUANTIFOIL Instruments GmbH) for 15 minutes at 37° C. at 1200 rpm. After digestion, the cleaned particles were washed twice with 100 μL of particle buffer and resuspended in 100 μL of particle buffer.

Cleaned particles were prepared for genotyping by exposing the bound DNA as single-stranded molecules (ssDNA particles). Particles were rotary mixed for 2 minutes at 95° C. at 1200 rpm to melt off the secondary strand of amplicon DNA that was not covalently bound to the hydrogel matrix. Only the strand originating from an extended acyrdite-modified primer remained in the particles as single-stranded DNA. The 95° C. incubation was followed immediately by centrifugation for 1 minute at 12,500 rpm to separate the particles from the aqueous solution containing the melted-off secondary strand. The particles were further washed twice with particle buffer at 95° C. After a final centrifugation the particles were not resuspended; only the pellet was retained.

In the first step that differentiated the wild-type from mutant DNA, the bound DNA in the wild-type ssDNA particles was selectively replicated, whereas the mutant ssDNA was unaffected. A primer specific to the variable region of the wild-type (variable primer) was annealed and extended for 2 minutes at 62° C. under 1200 rpm rotary mixing. The mutant particles lacked the intact variable domain sequence. Therefore, under the stringent hybridization conditions of the assay, no extension occurred along the mutant DNA template, leaving the mutant DNA in single-stranded form. The following conditions were used for selective extension: variable primer (SEQ ID NO: 10), 0.9 mM; dNTPs (New England Biolabs), 0.3 mM; Taq polymerase (New England Biolabs), 0.05 U/μL; and 1×Taq buffer. Extension was followed by two washes with 100 μL particle buffer at 62° C. After washing the particles were retained as a pellet.

Continuing the differentiation between mutant and wild-type DNA, the wild-type DNA was selectively trimmed from the particles by restriction enzyme digestion. A restriction enzyme specific to double-stranded DNA, BamHI (New England Biolabs), released the double stranded DNA from the wild-type particles but left the single-stranded DNA intact on the mutant particles. A BamHI site was incorporated into the acrydite primer sequence to present a cut site at a determined location near the anchor point to the gel. The following conditions were used for restriction enzyme digestion: particles were rotary mixed for 30 minutes at 37° C. at 1200 rpm with 1×NEBuffer 3.1 and 100 U/μL of BamHI enzyme. Digestion was followed by two washes with 100 μL of particle buffer, and particles were retained as a pellet. Note that in this example, a second BamHI site was also present in the sequence between the first BamHI site and the variable region. Thus the invention considers restriction sites that arise both naturally in the target DNA sequence and those that are engineered into the final construct. All combinations of natural, engineered, or natural and engineered restriction sites are considered. Furthermore, those practiced in the art will recognize that a minimum of one restriction site is necessary, but that the methods of the invention can accommodate multiple restriction sites of the same or difference sequences.

In the final step of the assay, the mutant DNA was returned to the original double-stranded form to maximize the signal of the fluorescence read-out. A second extension step was performed with a primer that hybridizes to the 3′ tail-end of the DNA bound to the particle. In this example, the reverse primer (SEQ ID NO: 9) for the original PCR was used, recovering the full length dsDNA of the original amplicon. However, the invention considers any reverse primer that yields dsDNA. Due to the endonuclease digestion, there is no wild-type DNA to extend and therefore only double-stranded mutant DNA is recovered. The following conditions were used for extension: particles were rotary mixed for 2 minutes at 62° C. at 1200 rpm with 0.9 mM primer, 0.3 mM dNTPs, 1×Taq buffer, and 0.05 U/μl Taq polymerase. Incubation was followed by two washes with 100 μL of particle buffer at 62° C., and the particles were retained as a pellet. The patent does consider that single-stranded DNA can be visualized directly as well without this last step, and any methods to visualize the mutant DNA are considered. For example, single-stranded DNA can be visualized by fluorescence detection after intercalation.

In a final confirmation that the mutant DNA that was recovered after the full assay was indeed double-stranded, a second exonuclease digestion was performed to eliminate any residual single-stranded DNA. Particles were rotary mixed for 15 minutes at 37° C. at 1200 rpm with 1× Exonuclease I buffer and 10 U/mL of Exonuclease I, followed by two washes with 100 μL of particle buffer. Particles were then resuspended in 100 μL of particle buffer for final non-specific fluorescence staining with 1× YOYO-1 (Thermo Fisher), diluted to an intermediate 100× working stock in DMSO from the original 10,000× commercial stock.

The fluorescence intensity of the particles was analyzed on a Guava easyCyte flow cytometer (EMD Millipore). Cytometry settings were established from the original particle sample before genotyping. Initially, the gain was set for the forward scatter to reveal a clear particle population. Particles were then gated by forward scatter, selecting only the single, dominant particle population that generally appeared for highly uniform particle populations. The original particles typically yielded high fluorescence signals compared to other assay intermediates and controls, and on this basis the green fluorescence gain—suitable for YOYO-1—was adjusted to accommodate the full spread of particle intensities.

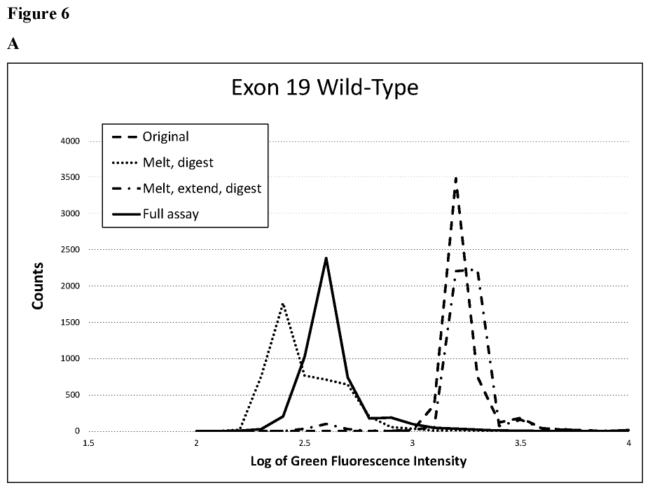

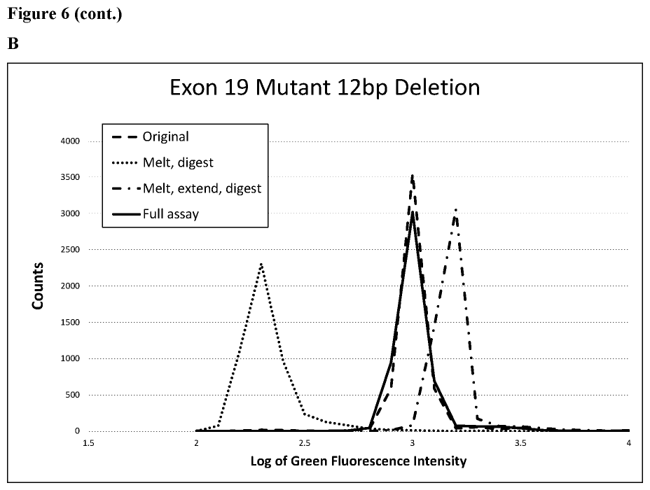

As set forth above, FIG. 6 shows of the log of the green fluorescence intensities of particles with wild-type and mutant DNA. For both the wild-type and the mutant particles the same trend is followed for the “original”, “melt, digest”, and “melt, extend, digest” particles, demonstrating that it is possible to knock down the fluorescence signal in particles and then regain it again through extension. These controls confirm that the polymerase and exonuclease enzyme activities are fully functional throughout the microgel matrix. The “full assay” results are different between the two genotypes. The wild-type particles exhibit a low fluorescence signal similar to the negative control, consistent with degradation of the wild-type DNA. In contrast, the mutant particles fluoresce brightly like the positive controls, confirming that the mutant DNA was protected as anticipated. Fluorescent images (IX81, Olympus) taken using fluorescence filters (ET480/40x excitation, ET535/50m emission, T5101pxrxt BS dichroic, Chroma) and high efficiency fluorescence imaging camera (Insight, SPOT Imaging) support these conclusions (data not shown). In conclusion, these methods of the invention succeeded in enrichment of indel-type mutant DNA by allele-specific degradation of wild-type DNA.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

What is claimed is:

1. A method comprising:
providing a nucleic acid target, the nucleic acid target comprising:
a locus of genetic variation, and
a conserved region on the 3′-side of the locus of genetic variation;
dissociating any associated strands within the nucleic acid target;
hybridizing an oligomer within the conserved region adjacent to the 3′-side of the locus of genetic variation;
extending the oligomer with a first chain terminating nucleotide that is sequence-specific to one genotype of the nucleic acid target, the extension reaction yielding:
a chain-terminated product if the sequence is matching, or
no affect if the sequence is mismatching;
replacing the first chain terminating nucleotide with a second mixture of extensible nucleotides;
extending the oligomer with the second mixture of extensible nucleotides, yielding:
no affect if the oligomer was chain-terminated, yielding a terminated oligomer, or
an extended oligomer product if the oligomer was extensible, yielding an extended oligomer.

2. The method of claim 1, wherein
the melting temperature of the complex between the nucleic acid target and the extended oligomer
is higher than
the melting temperature of the complex between the nucleic acid target and the terminated oligomer.

3. The method of claim 2 wherein the melting temperature of the complex between the nucleic acid target and the extended oligomer is at least five degrees Celsius different than the melting temperature of the complex between the nucleic acid target and the terminated oligomer.

4. The method of claim 2, wherein the temperature is poised
below the melting temperature of the complex between the nucleic acid target and the extended oligomer, yielding associated double-stranded DNA, and
above the melting temperature of the complex between the nucleic acid target and the terminated oligomer, yielding dissociated single-stranded DNA.

5. The method of claim 4, wherein the single-stranded DNA is degraded by enzymatic digestion, but the double-stranded DNA remains intact.

6. The method of claim 4, wherein the double-stranded DNA is detected with a DNA recognition agent or DNA recognition system.

7. The method of claim 6, wherein the DNA recognition agent is a fluorescent intercalating dye.

8. The methods of claim 1, wherein the nucleic acid target is tethered to a solid support.

9. The method of claim 8, wherein the solid support is a hydrogel.

10. The method of claim 9, wherein the hydrogel is a microparticle.

11. The method of claim 1, wherein the first chain terminating nucleotide is a dideoxynucleotide (ddNTP).

12-17. (canceled)

18. A method comprising:
providing a nucleic acid target, the nucleic acid target comprising a locus of genetic variation;
dissociating any associated strands within the nucleic acid target;
hybridizing an oligomer overlapping the locus of genetic variation, and yielding a double-stranded DNA complex with the nucleic acid target in the presence of the wild-type sequence, but not for genetic variants;
extending the oligomer, yielding:
an extended oligomer product for oligomers bound to the wild-type nucleic acid target wherein the oligomer product and the wild-type nucleic acid target for a double-stranded complex, or
no product for the mutant-type nucleic acid target.

19. The method of claim 18, wherein the nucleic acid targets are tethered to a solid support.

20. The method of claim 19, wherein the solid support is a hydrogel.

21. The method of claim 20, wherein the hydrogel is a microparticle.

22. The method of claim 18, wherein the extended oligomers products arising from nucleic acid targets with mutations are selectively released from the solid support by cleavage of the double-stranded DNA.

23. The method of claim 18, wherein the double-stranded DNA is cleaved by restriction enzyme digestion.

24. The method of claim 1, further comprising an additional step of melting, extending, or digesting, or combinations thereof, to maximize the signal of the nucleic acid target.

25-53. (canceled)

54. A method of diagnosing cancer in a subject, the method comprising the steps of:
determining in a biological sample of the subject enrichment levels of at least one nucleic acid target according to the method of claim 1;
wherein a significant modulation in the enrichment levels of said nucleic acid target in the sample is an indication that the subject is afflicted with cancer.

55. A method of prognosing cancer in a subject, the method comprising
the steps of:
determining in a biological sample of the subject enrichment levels of at least one nucleic acid target according to the method of claim 1;
wherein a significant modulation in the enrichment levels of said nucleic acid target in the sample is an indication that the subject has an unfavorable prognosis.

56. A method of treating a subject having cancer, the method comprising the steps of:
determining in a biological sample of the subject enrichment levels of at least one nucleic acid target according to the method of claim 1; and
providing a therapeutic treatment suitable to treat the cancer.

57. The method of claim 56, wherein the cancer is selected from the group consisting of hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

58. The method of claim 56, wherein the therapeutic treatment is selected from the group consisting of surgery, immunotherapy, chemotherapy, radiation therapy, a combination of chemotherapy and radiation therapy, and biological therapy.

59. The method of claim 56, wherein the sample is selected from the group of consisting of a tumor sample, tissue, histological slides, frozen core biopsies, paraffin embedded tissues, formalin fixed tissues, biopsies, blood, urine, plasma, and saliva.

60. The method of claim 18, further comprising an additional step of melting, extending, or digesting, or combinations thereof, to maximize the signal of the nucleic acid target.

61. A method of diagnosing cancer in a subject, the method comprising the steps of:
determining in a biological sample of the subject enrichment levels of at least one nucleic acid target according to the method of claim 18;
wherein a significant modulation in the enrichment levels of said nucleic acid target in the sample is an indication that the subject is afflicted with cancer.

62. The method of claim 61, wherein the cancer is selected from the group consisting of hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

63. The method of claim 61, wherein the therapeutic treatment is selected from the group consisting of surgery, immunotherapy, chemotherapy, radiation therapy, a combination of chemotherapy and radiation therapy, and biological therapy.

64. The method of claim 63, wherein the sample is selected from the group of consisting of a tumor sample, tissue, histological slides, frozen core biopsies, paraffin embedded tissues, formalin fixed tissues, biopsies, blood, urine, plasma, and saliva.

65. A method of prognosing cancer in a subject, the method comprising the steps of:
determining in a biological sample of the subject enrichment levels of at least one nucleic acid target according to the method of claim 18;
wherein a significant modulation in the enrichment levels of said nucleic acid target in the sample is an indication that the subject has an unfavorable prognosis.

66. The method of claim 65, wherein the cancer is selected from the group consisting of hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

67. The method of claim 65, wherein the therapeutic treatment is selected from the group consisting of surgery, immunotherapy, chemotherapy, radiation therapy, a combination of chemotherapy and radiation therapy, and biological therapy.

68. The method of claim 67, wherein the sample is selected from the group of consisting of a tumor sample, tissue, histological slides, frozen core biopsies, paraffin embedded tissues, formalin fixed tissues, biopsies, blood, urine, plasma, and saliva.

69. A method of treating a subject having cancer, the method comprising the steps of:
determining in a biological sample of the subject enrichment levels of at least one nucleic acid target according to the method of claim 18; and
providing a therapeutic treatment suitable to treat the cancer.

70. The method of claim 69, wherein the cancer is selected from the group consisting of hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

71. The method of claim 69, wherein the therapeutic treatment is selected from the group consisting of surgery, immunotherapy, chemotherapy, radiation therapy, a combination of chemotherapy and radiation therapy, and biological therapy.

72. The method of claim 71, wherein the sample is selected from the group of consisting of a tumor sample, tissue, histological slides, frozen core biopsies, paraffin embedded tissues, formalin fixed tissues, biopsies, blood, urine, plasma, and saliva.
